(19) United States
(12) Patent Application Publication
(10) Pub. No.: US 2026/0286292 A1
(43) Pub. Date: Sep. 24, 2026

(54) METHODS FOR LARGE SCALE EXPANSION OF PLURIPOTENT STEM CELLS (PSCS)

(71) Applicant: STEMCELL TECHNOLOGIES CANADA INC., Vancouver (CA)

(72) Inventors: Olivia NEEF, Vancouver (CA); Kimberly SNYDER, Vancouver (CA); Arwen HUNTER, Vancouver (CA); Adam HIRST, Vancouver (CA); Matthew WONG, Vancouver (CA)

(21) Appl. No.: 19/473,529

(22) PCT Filed: Apr. 19, 2024
(86) PCT No.: PCT/CA2024/050519

§ 371 Date: Oct. 8, 2025

Related U.S. Application Data
(60) Provisional application No. 63/460,751, filed on Apr. 20, 2023.

(51) Int. Cl. C12N 5/0735; C12N 5/074

(52) U.S. Cl. CPC C12N 5/0606; C12N 5/0696; C12N 2533/90

(57) ABSTRACT

The present disclosure relates to cell culture applications, and more specifically to cell culture applications for expanding cells, such as stem cells (e.g. PSC). Methods include seeding cells in a first vessel and optionally transferring cells from the first vessel to a second vessel. Cells of the first vessel, and as applicable in the second vessel, are contacted with at least one exogenously added extracellular matrix or extracellular matrix protein. Methods of this disclosure yield significantly and rapidly expanded populations of cells, while maintaining cell quality. Methods of this disclosure may also improve cell plating efficiencies of cells to be significantly and rapidly cultured/expanded in monolayer.

### CROSS-REFERENCE TO RELATED APPLICATION

This application claims the benefit of U.S. Provisional Patent Application No. 63/460,751, filed Apr. 20, 2023, the entire content of which is hereby incorporated by reference in its entirety.

### TECHNICAL FIELD

This disclosure relates to cell culture applications, and more specifically to cell culture applications using stem cells, and still more specifically to cell culture applications related to stem cells such as pluripotent stem cells (PSC).

### BACKGROUND

Scaling up and/or speeding up cell culture expansion protocols is of interest in many applications, including for cell therapy applications, protein production applications, and regenerative medicine applications, to name a few. Such applications require large quantities of high-quality cells that can be reproducibly obtained in a cost-effective manner.

Pluripotent and other stem cells, with their unique characteristics of self-renewal and ability to differentiate into downstream lineages, are uniquely poised to revolutionize the fields of cell therapy, drug discovery, disease modeling and regenerative medicine. Use of cells in clinical trials, in particular, requires incredibly large numbers of cells per patient dose.

Thus, there remains an unmet need for efficient and scalable methods for manufacturing cells, such as stem cells, in large quantities and at high-quality to meet growing commercial and clinical needs.

### SUMMARY

The present disclosure relates to methods for scaling expansion of cells, such as in a monolayer. The methods may relate to stem cells, such as pluripotent stem cells.

In one aspect of this disclosure are provided methods of expanding cells, such as PSC, in monolayer. Methods of this disclosure may comprise providing a first suspension of cells, such as in a cell culture medium or other supportive liquid, depositing cells of the first suspension into a first vessel, and culturing the deposited cells as a monolayer in a cell culture medium.

Methods of this disclosure may further comprise (prior to culturing the deposited cells), contacting the deposited cells with at least one exogenously added extracellular matrix or extracellular matrix protein. In one embodiment, the first vessel is not pre-coated with the at least one exogenously added extracellular matrix or extracellular matrix protein.

Extracellular matrices or extracellular matrix proteins of this disclosure are not particularly limited, but in some embodiments may be Matrigel™ or the like, or an isolated or purified extracellular matrix protein, such as fibronectin, vitronectin or a laminin isoform, or a fragment thereof.

Methods of this disclosure may further comprise dissociating the monolayer (of cells) and preparing a second suspension of cells, such as in a cell culture medium or other supportive liquid.

Methods of this disclosure may further comprise depositing cells of the second suspension into a second vessel, and culturing the deposited cells as a monolayer in a cell culture medium. Methods of this disclosure may further comprise (prior to culturing such deposited cells), contacting the deposited cells with at least one exogenously added extracellular matrix or extracellular matrix protein. In one embodiment, the second vessel is not pre-coated with the at least one exogenously added extracellular matrix or extracellular matrix protein.

In methods of this disclosure, the deposited cells may be contacted with the at least one exogenously added extracellular matrix or extracellular matrix protein before, after, or at the same time as being deposited into the first or second vessel.

In another aspect of this disclosure are provided methods (e.g. expedited methods) of expanding cells, such as PSC, in monolayer. Methods of this disclosure may comprise providing a first suspension of cells, such as in a cell culture medium or other supportive liquid, depositing cells of the first suspension into a first vessel, culturing the deposited cells as a monolayer in a cell culture medium, dissociating the monolayer and preparing a second suspension of cells, and depositing and culturing cells of the second suspension of cells essentially as described in the foregoing to yield greater than 1000-fold expansion in two weeks or less.

Methods of this aspect of the disclosure may further comprise (prior to culturing the deposited cells), contacting the deposited cells with at least one exogenously added extracellular matrix or extracellular matrix protein. In one embodiment, the first and/or the second vessel is not pre-coated with the at least one exogenously added extracellular matrix or extracellular matrix protein.

In another aspect of this disclosure are provided methods of improving cell plating efficiencies of cells to be cultured in monolayer. Methods of this disclosure may comprise providing a first suspension of cells, such as in a cell culture medium or other supportive liquid, depositing cells of the first suspension into a first vessel, and culturing the deposited cells as a monolayer in a cell culture medium.

Methods of this disclosure may further comprise (prior to culturing the deposited cells), contacting the deposited cells with at least one exogenously added extracellular matrix or extracellular matrix protein. In one embodiment, the first vessel is not pre-coated with the at least one exogenously added extracellular matrix or extracellular matrix protein.

Methods of this aspect of the disclosure may further comprise dissociating the monolayer (of cells) and preparing a second suspension of cells, such as in a cell culture medium or other supportive liquid.

Methods of this aspect of the disclosure may further comprise depositing cells of the second suspension into a second vessel, and culturing the deposited cells as a monolayer in a cell culture medium. Methods of this aspect of the disclosure may further comprise (prior to culturing such deposited cells), contacting the deposited cells with at least one exogenously added extracellular matrix or extracellular matrix protein. In one embodiment, the second vessel is not pre-coated with the at least one exogenously added extracellular matrix or extracellular matrix protein.

In another aspect of this disclosure are provided methods of improving cell plating efficiencies of cells to be cultured in monolayer. Methods of this aspect of the disclosure may comprise providing a first suspension of cells, such as in a cell culture medium or other supportive liquid, depositing cells of the first suspension into a first vessel, culturing the deposited cells as a monolayer in a cell culture medium, dissociating the monolayer and preparing a second suspension of cells, and depositing and culturing cells of the second suspension of cells essentially as described in the foregoing such as to yield greater than 1000-fold expansion in two weeks or less.

In any aspect, extracellular matrices or extracellular matrix proteins of this disclosure are not particularly limited, but in some embodiments may be Matrigel™ or the like, or an isolated or purified extracellular matrix protein, such as fibronectin, vitronectin or a laminin isoform, or a fragment thereof. In embodiments of any aspect, the at least one exogenously added extracellular matrix protein is recombinant, or the extracellular matrix is a mixture of two or more extracellular matrix proteins.

In any aspect, a (total or combined) culture surface area of the second vessel may be greater than a (total or combined) culture surface area of the first vessel. In one embodiment, a culture surface area of the second vessel is at least 10 times greater than a culture surface area of the first vessel.

In any aspect, methods of this disclosure may further comprise dissociating the monolayer from the second vessel to yield a third suspension. A quantity of cells in the second (or third) suspension exceeds a quantity of cells in the first suspension by at least 3 orders of magnitude, which culture period may elapse two weeks or less, such as 14 days, 13 days, 12, days, 11, days, 10 days, 9 days, 8 days, 7 days, 6 days, or 5 days.

Methods of this disclosure may further comprise contacting deposited cells (such as by supplementing a culture medium) with an attachment supplement. In one embodiment, the attachment supplement comprises an albumin and one or more fatty acids. Contact with an attachment supplement may increase plating efficiency of deposited cells.

Through contact with the attachment supplement and/or the at least one exogenously added extracellular matrix or extracellular matrix protein, a greater percentage of deposited cells may adhere to a wall of the first vessel or the second vessel, respectively, when contacted with the attachment supplement and/or the at least one exogenously added extracellular matrix or extracellular matrix protein than if deposited without coming into contact with the at least one exogenously added extracellular matrix or extracellular matrix protein.

Through contact with the attachment supplement and/or the at least one exogenously added extracellular matrix or extracellular matrix protein, a greater percentage of deposited cells of one or both of the first suspension and the second suspension may adhere respectively to a wall of the first vessel and a wall of the second vessel when seeded in the presence of the culture medium and the attachment supplement and/or the at least one exogenously added extracellular matrix or extracellular matrix protein than if seeded in the culture medium lacking the attachment supplement and/or the at least one exogenously added extracellular matrix or extracellular matrix protein.

Through contact with the attachment supplement and/or the at least one exogenously added extracellular matrix or extracellular matrix protein, a greater than 1000-fold expansion is expedited when culturing the deposited cells in contact with the culture medium and in the presence of the attachment supplement and/or the at least one exogenously added extracellular matrix or extracellular matrix protein in comparison to culturing the deposited cells in contact with the culture medium and not in the presence of the attachment supplement and/or the at least one exogenously added extracellular matrix or extracellular matrix protein.

Through contact with the attachment supplement and/or the at least one exogenously added extracellular matrix or extracellular matrix protein, a greater than 1000-fold expansion is expedited when culturing the deposited cells of the first suspension in contact with the cell culture medium and the attachment supplement and/or the at least one exogenously added extracellular matrix or extracellular matrix protein in comparison to culturing the deposited cells of the first suspension in contact with the culture medium but not the attachment supplement and/or the at least one exogenously added extracellular matrix or extracellular matrix protein.

In methods of any aspect of the disclosure, the deposited cells may be contacted with the at least one exogenously added extracellular matrix or extracellular matrix protein before, after, or at the same time as being deposited into the first or second vessel.

In methods of any aspect of the disclosure, the deposited cells may be contacted with the attachment supplement before, after, or at the same time as being deposited into the first or second vessel.

Methods of this disclosure may further comprise combining at least one exogenously added extracellular matrix or extracellular matrix protein and a culture medium before the deposited cells are contacted with the at least one exogenously added extracellular matrix or extracellular matrix protein and/or the attachment supplement.

Methods of this disclosure may further comprise spiking or adding at least one extracellular matrix or extracellular matrix protein and/or the attachment supplement into a cell culture medium, which medium may be pre-dispensed into the first or second vessel.

In any aspect of this disclosure, the methods may be automated.

Other features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

For a better understanding of the various embodiments described herein, and to show more clearly how these various embodiments may be carried into effect, reference will be made, by way of example, to the accompanying drawings which show at least one example embodiment, and which are now described. The drawings are not intended to limit the scope of the teachings described herein.

FIG. 1 shows expansion of STi0003-A PSC in monolayer. Representative microscopic images at 20× and 100× magnification show morphology and day-5 expansion levels of cells cultured in contact with extracellular matrix either pre-coated or not pre-coated (“spike-in”) on a vessel. In both conditions the extracellular matrix was present at either 4 μg/cm² or 9 μg/cm² (A). Bar graphs show day-5 fold expansion (B) and viable cell count (C) of cells either pre-coated or non-pre-coated (spiked-in) Matrigel™ at 4, 5, 6, 7, 8, and 9 μmg/cm². For (B), the data is normalized to 9 μg/cm² Matrigel™ Scale bars represent 100 μm and 500 μm for 100× and 20× magnification, respectively.

FIG. 2 shows bar graphs characterizing day-5 expanded cells cultured in accordance with coating-free methods of this disclosure, in terms of viability (A), cell yield per mL media (B), and fold expansion (C). Bars represent mean±SD of 7 biological replicates.

FIG. 3 shows assessments of cell quality among expanded cells. Bar graph quantifying the expression of undifferentiated markers OCT4 and TRA-1-60 among day-5 expanded PSCs (SCTi003-A, WLS-1C, and H9) cultured in accordance with coating-free methods of this disclosure (A). Data is shown as mean±SD of 2 technical replicates. Bar graph of percent recovery (viable cells harvested/viable cells seeded×100) of cells expanded in accordance with coating-free methods of this disclosure in a scale-out vessel compared to a matched companion plate of cells deposited in Matrigel™ pre-coated 96-well plates (12 wells per condition) (B). Data is shown as mean±standard deviation of 2 technical replicates.

FIG. 4 shows bar graphs summarizing post-thaw % viability of day-0 (A) and day-5 cells (B), and fold expansion of day-5 post-thaw cells (C). Thawed cells had been cultured in accordance with the coating-free methods of this disclosure. Data is shown as mean±SD.

FIG. 5 shows a graph quantifying clump counts of H9 cells when cultured in head-to-head experiments where different extracellular matrices or extracellular matrix proteins were pre-coated or not pre-coated (spike-in). Data is shown as mean±SD of 4 technical replicates.

FIG. 6 shows maintenance of SCTi003-A, WLS-1C, H9 and STIPS-R038 cells in mTeSR™ plus or TeSRIM-AOF where Matrigel™ is either pre-coated on culture vessels or cultured in accordance with coating-free methods of this disclosure. Representative microscopic images at 2× magnification show colony morphology after 10-20 passages. Scale bars represent 500 μm. Flow cytometry analysis of % positive expression of markers of the undifferentiated state (OCT4 & TRA160) after 10-20 passage of cells cultured in mTeSR™ Plus or TeSR™-AOF in pre-coating conditions, coating-free conditions of this disclosure alone or in combination with a polymeric surfactant. Bars represent mean±SD of 2 biological replicates for WLS-1C and STIPS-R038 cells and 4 biological replicates for H9 and SCTi003-A cells.

FIG. 7 quantifies characteristics of cells cultured in accordance with coating-free methods of this disclosure. Cells were cultured in mTeSR™ Plus media for 10-20 passages using enzymatic dissociation (A) or 1-3 passages using ReLeSRT-based scrape-dissociation (B) after culture in either pre-coated Matrigel™ plates (PC), coating-free conditions of this disclosure (CF), or coating-free conditions together with a polymeric surfactant (CF+S). Plating efficiency of the cells cultured as described in (A) and (B) in either mTeSR™ plus (C) or TeSR™-AOF (D). Plating efficiency was calculated by dividing the total number of clumps seeded by the total number of colonies at the end of the passage.

FIG. 8 shows the trilineage differentiation potential of cells cultured in mTeSR™ plus media (A, C & E) under pre-coated Matrigel™ conditions (PC) and coating-free conditions with and without surfactant. Trilineage differentiation potential of cells cultured in TeSR™-AOF media under the same conditions as in (A), (C), and (E) (B, D & F). Bar graphs quantify % positive cells for ectoderm markers Nestin & PAX6 (8A & 8B), mesoderm markers NCAM & Brachyury (8C & 8D) and endoderm markers SOX17 & CXCR4 (8E & 8F). Data is shown as mean±SD deviation. Each bar represents 2 technical replicates.

FIG. 9 shows morphology and expansion of porcine PSCs (pPSCs) cultured with spiked-in LN511 compared to a LN511 pre-coated control. Representative microscopic images at 4× magnification show morphology of pPSCs at the end of P 3 on day 5. 4000 cells were seeded in each condition. LN511 was added to cultures at 50%, 75% and 100% of the LN511 concentration (0.375 mg/ml) of pre-coated plates.

FIG. 10 shows morphology and expansion of pPSCs cultured with spiked-in LN521 compared to a LN511 pre-coating control. Representative microscopic images show morphology of pPSCs captured at the end of PO. LN511 was pre-coated at a concentration (0.375 mg/ml) whereas LN521 was added to the cultures at: 1 μg/ml, 2.5 μg/ml, 5 μg/ml, 7.5 μg/ml and 10 μg/ml.

### DETAILED DESCRIPTION

This disclosure relates to media compositions, supplements, and methods for expanding cells, such as in monolayer. More specifically, this disclosure relates to expanding cells, such as in monolayer, in the presence of at least one exogenously added extracellular matrix or extracellular matrix protein, as may be added to or contacted with a cell suspension.

Where used in this disclosure, the term “stem cell” refers to a cell that is capable of self-renewal and differentiation to at least one downstream lineage. Where specifically used in this disclosure, the term “pluripotent stem cell” or “PSC”, or pluralized variations, refers to a cell that is capable of self-renewal and of differentiating to all three germ layers, and beyond. The conditions and media requirements for culturing stem cells, and in the case of PSC to maintain them in an undifferentiated state, are largely known but improvements to these conditions are the subject of ongoing research. PSC encompass both embryonic stem cells (“ESC”) and induced pluripotent stem cells (“iPSC”), and the like. Stem cells, such as PSC, are an important model to study differentiation mechanisms, to model diseases, and offer significant medical opportunities. Stem cells may be obtained, derived or induced from any source species, but in this disclosure, stem cells are preferably mammalian.

Where used in this disclosure, the term “extracellular matrix”, “extracellular matrix protein” or “ECM” refers to one or more molecule(s) produced or secreted by cells that provide structural and biochemical support to cells, such as by promoting cell adhesion, cell-to-cell communication, and/or differentiation within a given tissue or culture of cells. An extracellular matrix may comprise an interlocking mesh of fibrous proteins and glycosaminoglycans. In the context of this disclosure, when culturing mammalian cells (e.g., stem cells or PSC) in monolayer, whether seeded as single cells or as clusters, it may be desirable or necessary to do so in the presence of a suitable matrix or matrix protein, which may be comprised in a cell culture medium or a cell suspension (in cell culture medium or other supportive liquid such as a buffer). The matrix or matrix protein may be any that supports expansion of the one or more mammalian stem cells, and both natural and synthetic extracellular matrices or proteins are contemplated within the present disclosure. An extracellular matrix may comprise one or more extracellular matrix proteins. In one embodiment, an extracellular matrix is a mixture of two or more extracellular matrix proteins. Components of an extracellular matrix, and thus an extracellular matrix within the scope of this disclosure, may include one or more of the following proteins: a collagen, a fibronectin, a laminin, and a vitronectin. An extracellular matrix used in or with a culture medium described herein may be a gelatinous protein mixture secreted by cells, such as fibroblasts, chondrocytes, or Engelbreth-Holm-Swarm (EHS) mouse sarcoma cells, such as Matrigel™. The one or more extracellular matrix proteins may be recombinant, and/or may be fusion proteins. In one embodiment, extracellular matrix protein(s) of this disclosure are human and/or mouse proteins, or fragments thereof. In one embodiment, the one or more exogenously added extracellular matrix proteins may be of natural origin. In one embodiment, the one or more extracellular matrix proteins may be genetically engineered. In one embodiment, the one or more extracellular matrix proteins may be a whole protein or a fragment thereof, such as a peptide fragment.

Where used in this disclosure, the term “cell culture medium” refers to a liquid, semi-liquid or gelatinous substance containing nutrients in which cells or tissues can be cultivated (e.g., expanded and/or maintained). Cell culture media typically supply components required by cells to meet nutritional needs as they proliferate, differentiate, or are maintained in vitro. Cell culture media of this disclosure may be serum-free and/or xeno-free and/or animal component-free. In addition cell culture media of this disclosure may be used in a feeder cell free conditions or environments. Cell culture media of this disclosure may comprise a basal medium. Basal media may require supplementation with one or more additives to formulate a complete medium. In one embodiment, PSC may be maintained and/or expanded in a commercially available cell culture medium, such as in a TeSR™ branded formulation (e.g., mTeSR™1, TeSR™-AOF, TeSR™-E8, or mTeSR™ Plus). Other commercially available media for supporting stem cell growth/expansion are known. In one embodiment, porcine PSCs may be maintained and/or expanded in a porcine PSC medium comprising a WNT pathway inhibitor, an FGF and a member of the transforming growth factor beta (TGF-β) family of proteins. In one embodiment, the porcine PSC medium may comprise Activin A, FGF and XAV939.

Where used in this disclosure, the term “attachment supplement” refers to a cell culture supplement that facilitates initial and/or continued adherence of cells to the surface of cell culture containers (e.g., cell culture flasks and other cell culture vessels). An attachment supplement may comprise a carrier, such as by way of non-limiting example, an albumin, a micelle, a liposome, an extracellular vesicle, an exosome, a cyclodextrin, a nanostructured lipid carrier, or otherwise. In one embodiment, a carrier is or comprises a lipid-containing or lipid-enriched albumin. In embodiments where the carrier is an albumin, the albumin may be from any source (e.g., BSA, HSA, recombinant, or otherwise), and certain albumins may be better suited for culturing stem cells. Attachment supplements comprising a lipid-containing or enriched carrier, may comprise one or more lipids/fatty acids or more than one lipid/fatty acid. By way of non-limiting example, one or more lipids/fatty acids or more than one lipid/fatty acid may be one or more of Mead's acid, arachidic acid, palmitoleic acid, oleic acid, myristic acid, palmitic acid, myristoleic acid, linoleic acid, stearic acid, a-linolenic acid, arachidonic acid, cholesterol, DL-a-tocopheryl, and Kolliphor P188.

Where used in this disclosure, the term “spiked-in” or “spiked”, used interchangeably with the term “exogenously added” or coating-free, refers to an extracellular matrix, or one or more extracellular matrix proteins, having been added directly to a cell suspension or a cell culture medium. In one embodiment, a desired quantity or concentration of the matrix or one or more proteins may be added to a cell culture medium comprising a population or suspension of cells. In one embodiment, a desired quantity or concentration of matrix or one or more proteins may be added to a cell culture medium that later comes into contact with a population or suspension of cells. In one embodiment, a desired quantity or concentration of a matrix or one or more proteins may be added to a cell culture vessel containing a cell culture medium or a cell suspension, or both.

### Methods

In one aspect of this disclosure are provided methods of expanding cells, such as stem cells (e.g. PSC), optionally in a monolayer. Cells of this disclosure may be expanded in a large-scale cell culture vessel, such as a Cell Factory™ or a CELLSTACK™ (e.g. a “scale-out vessel”).

Methods of this disclosure may comprise providing a first suspension of cells. The first suspension of cells may be provided in a cell culture medium, as a pellet, or in another supportive liquid such as in a buffer.

A first suspension of cells is not particularly limited and may comprise any cell type, such as a stem cell, capable of expansion in culture. Stem cells, such as a MSCs, epithelial stem cell, neural stem cells, HSPC, PSC, or the like, are capable of self-renewal in appropriate culture conditions.

Certain embodiments of this disclosure relate to PSC, thus the first suspension of stem cells may be a suspension of PSC, such as ESC or iPSC. Stem cells, such as PSC, may be derived from any animal or mammalian species, including human, primate, rodent or livestock source. In one embodiment, the PSC are human PSC (hPSCs), such as human ESC (hESC) or human iPSC (hiPSC). In one embodiment, the PSC are livestock-derived PSC, such as porcine PSC (pPSC), bovine PSC, hircine PSC, caprine PSC or ovine PSC.

A first suspension of stem cells may be obtained from an appropriate cell culture vessel, such as one or more wells of a 96-, 48-, 24-, 12-, or 6-well plate, or one or more dishes, such as 35 mm or 10 cm dishes. Typically, a single well of a 6-well plate at 75-95% confluency contains between approximately 1×10⁶ and 4×10⁶ cells (e.g. stem cells), or between approximately 2×10⁶ and 3×10⁶ of such cells.

Methods of this disclosure may further comprise seeding or depositing cells of the first suspension into a first vessel. The cells of the first suspension deposited or seeded in the first vessel may be seeded as single cells, or as clumps of cells comprising about 3, about 5, about 8, about 10, about 15, about 20, about 50, about 100, or more cells.

Cells (of the first suspension) may be deposited or seeded into the first vessel at an appropriate density taking into consideration the culturing surface area of the first vessel and the quantity of cells in the first suspension. The seeding density may range between about 5000 and 15000cells/cm² , between about 7000 and 13000cells/cm² , or between about 8500 and 11500cells/cm² . More specifically, the seeding density may range between about 9000 cells/cm²±1000 cells, or may range between 10000 cells/cm²±1000 cells, or more.

The nature of the first vessel is not particularly limited. In some cases it may be important that that a culturing surface area of the first vessel is greater than a culturing surface area of the cell culture vessel from which the first suspension was obtained. For example, where the cell culture vessel from which the first suspension was obtained is a well of 6-well dish or the like, the first vessel may be a T-150 or a T-175 flask. Alternatively, and by way of non-limiting example, if the cell culture vessel from which the first suspension was obtained is each well of a 6-well dish, or the like, the first vessel will have a greater culture surface area than the combined surface area of the parent culture, and potentially a 5×, 10×, 15×, 20× or greater culture surface area. The foregoing discussion is not limited to 6-well plate formats, and encompasses other plate or flask formats. Exemplary first (or second) vessels may include cell stacks or cell hotels, as are commercially available through Nunc™ or Corning™.

Methods of this disclosure may further comprise contacting the deposited cells (of the first suspension) with at least one exogenously added extracellular matrix or extracellular matrix protein. Preferably, the first vessel is not pre-coated with the at least one exogenously added extracellular matrix or extracellular matrix protein. However, in certain cases (such as certain stages of the coating-free methods disclosed herein), a first vessel may be pre-coated with at least one or more exogenously added extracellular matrix or extracellular matrix protein.

Extracellular matrices or extracellular matrix protein(s) of this disclosure, whether contacted with or by the deposited cells before, after, or at the same time as the cells are deposited into a (first) vessel, are not particularly limited. Non-limiting examples of at least one exogenously added extracellular matrix protein include a vitronectin, a laminin isoform, and/or a vitronectin, or the like, or a fragment of the foregoing. Non-limiting examples of at least one exogenously added extracellular matrix include Matrigel™ or the like, as may be produced by a population of cells (e.g. by Engelbreth-Holm-Swarm (EHS) mouse sarcoma cells). At least one exogenously added extracellular matrix or extracellular matrix protein may be a single matrix or protein. Alternatively, at least one exogenously added extracellular matrix or extracellular matrix protein may be a mixture of more than one matrix or protein.

A concentration of the extracellular matrix or the extracellular matrix protein(s) is not particularly limited, provided that it is not toxic or otherwise negatively impacts cell expansion/proliferation, and/or does not cause gelation of the cell culture medium. Depending on the nature of the matrix or protein(s) it may be more appropriate to relay the concentration in terms of a percentage (dilution) or a density. For example, a concentration of an extracellular matrix or extracellular matrix protein may be relative to a volume of the cell culture medium (such as of the first suspension of stem cells), such as between about 0-20% (v/v), or between about 0.05-10%, or between about 0.05-5%, or between about 0.1 and 1%. Alternatively, a concentration of an extracellular matrix or extracellular matrix protein may be represented as a dilution, such as for Matrigel™ or the like, and the dilution is about 1:50, 1:100, 1:150, 1:200, 1:250, 1:300 or more. Alternatively, a concentration of an extracellular matrix or extracellular matrix protein may range between about 0.1 μg/mL to 1 mg/mL, between about 1 μg/mL to 500 μg/mL, between about 3 μg/mL to 200 μg/mL, between about 5 μg/mL to 150 μg/mL, or between about 10 μg/mL to 100 μg/mL. Alternatively, a concentration of an extracellular matrix or extracellular matrix protein may range between about 1 μg/mL to 5 μg/mL.

In some cases, at least one exogenously added extracellular matrix or extracellular matrix protein is added to the first cell suspension before cells of the first suspension are deposited into the first vessel. Alternatively, at least one exogenously added extracellular matrix or extracellular matrix protein is added to cell-culture medium pre-dispended into the first cell culture vessel before the cells of the first cells suspension come into contact therewith. Alternatively, cells deposited in first cell culture vessel may subsequently come into contact with at least one exogenously added extracellular matrix or extracellular matrix protein by spiking or adding the matrix or protein.

Methods of this disclosure may further comprise culturing the cells contacted with the at least one exogenously added extracellular matrix or extracellular matrix protein as a monolayer in a cell culture medium.

A cell culture medium of this disclosure is not particularly limited, provided it supports the expansion of the cells (e.g. the self-renewal of the stem cells). Different media formulations are commercially available and are tailored for specific cell types. However, media optimized for specific cell types (e.g. XVIVO media) may not be well suited for other cell types (e.g. PSC). Exemplary PSC cell culture media are well known in the art and include those media sold by STEMCELL Technologies under the TeSR™ brand.

Deposited cells, as may be contacted with at least one exogenously added extracellular matrix or extracellular matrix protein, may be cultured for an appropriate length of time to yield a confluency between about 60% and 100%, or between about 70% and 95%. Provided that a seeding density appropriate for the cells and the surface area of the first vessel is used (e.g., between about 7500-12500 cells/cm²), a desired confluency may be reached in less than 1 week, in about 6 days or less, in about 5 days or less, in about 4 days or less, or in about 3 days or less.

Methods of this disclosure may further comprise dissociating a monolayer of cells (e.g. stem cells) from a first vessel and preparing a second suspension of cells.

An adhered monolayer of cells, such as stem cells, may be dissociated using conventional reagents and methodologies, for example by dissociation using an enzyme or a chemical reagent. Non-limiting examples of enzymatic dissociation reagents include trypsin-containing solutions, such as Accutase™ or TrypLE™. Non-limiting examples of chemical dissociation reagents may include chelators, such as EDTA or commercially available reagents such as ReLeSR™.

In methods of any aspect of this disclosure, the at least one exogenously added extracellular matrix or extracellular matrix protein may further comprise a surfactant. Without being bound by theory, contacting monolayers of this disclosure (whether in a first vessel, a second vessel, or otherwise) with the at least one exogenously added extracellular matrix or extracellular matrix protein comprising a surfactant may improve dissociation. Surfactants are known, and in certain cases may be polymers, copolymers, or block copolymers. By way of non-limiting example, a surfactant of this disclosure may be an ionic or a non-ionic polymer, copolymer, or block copolymer comprising Pluronic F-68, Pluronic F-127, Pluronic F-108 or Polyethylene glycol (PEG). Surfactants of this disclosure may be used at a concentration ranging from 0.001% to 10%, or at a concentration ranging from 0.01%-1%, or at a concentration in the range of about 0.05%±0.03%.

After contacting an adhered monolayer with a proteolytic or chemical dissociation reagent, the cells may be incubated in the reagent to dissociate the adhered monolayer of cells. Gentle tapping and/or trituration may help to dissociate the adhered monolayer of cells to yield a second suspension of cells. Alternatively, scraping using a cell scraper or a micropipette/pipette tip may help to dissociate and collect the cells within a well or a vessel to yield a second suspension of cells.

A second suspension of cells, prepared as described in the foregoing, may be resuspended in an appropriate liquid, such as a buffer or a cell culture medium, as described herein. The second suspension may comprise an expanded population of cells, such as where the first vessel is a scale-out vessel.

However, the second suspension may not be the final expanded population of cells where further expansion is desired. In such cases, methods of this disclosure may further comprise depositing or seeding cells of the second suspension into a second vessel. Cells of the second suspension may be deposited or seeded as single cells or as clumps of cells (as described above).

Also as described above, cells of the second suspension will be deposited or seeded into the second vessel at an appropriate density, taking into consideration the cell type, the number of cells in the second suspension of cells, and the nature of the second vessel. By way of example, cells of the second suspension may be seeded in the second vessel at a density between about 5000 and 15000cells/cm², between about 7000 and 13000 cells/cm², or between about 8500 and 11500cells/cm². In one embodiment, the seeding density is in the range between 9000 cells/cm²±1000 cells, or between 10000 cells/cm²±1000, or more.

Second vessels of this disclosure are not particularly limited, provided they have a greater culture surface area than the first vessel. For example, a culture surface area of the second vessel may be at least 5, 10, 15, 20, 50, 100 or 200 times greater than a culture surface area of the first vessel. More specifically, a culture surface area of the second vessel may be about 10 times greater than a culture surface area of the first vessel. Where the first vessel is each well of a 6-well plate, a T-150, or a T-175 flask, the second vessel may be a further scale-out vessel, such as is commercially available (a Nunc™ Cell Factory™ or a Corning™ CELLSTACK™, for example). The foregoing discussion is not limited to 6-well plate formats and cell stack or cell hotels, and is merely illustrative of scaled-up vessels.

Essentially as described above, with respect to cells of the first suspension deposited into the first vessel, cells of the second suspension deposited into the second vessel may be contacted with at least one exogenously added extracellular matrix or extracellular matrix protein. Description of the at least one exogenously added extracellular matrix or extracellular matrix protein coming into contact with cells of the second suspension, is as described herein, in terms of type, concentration, nature of the contact, coating or spiking, etc.

In some cases, at least one exogenously added extracellular matrix or extracellular matrix protein is added to the second cell suspension before cells of the first suspension are deposited into the second vessel. Alternatively, at least one exogenously added extracellular matrix or extracellular matrix protein is added to cell-culture medium pre-dispended into the second cell culture vessel before the cells of the second cell suspension come into contact therewith. Alternatively, cells deposited in second cell culture vessel may subsequently come into contact with at least one exogenously added extracellular matrix or extracellular matrix protein by spiking or adding the matrix or protein.

Further, a second vessel may not be pre-coated with at least one exogenously added extracellular matrix or extracellular matrix protein. Thus, in some embodiments, neither the first vessel nor the second vessel is pre-coated with at least one exogenously added extracellular matrix or extracellular matrix protein.

Methods of this disclosure may further comprise culturing cells of a second suspension (contacted with at least one exogenously added extracellular matrix or extracellular matrix protein) as a monolayer in a cell culture medium, as described above.

Cells of a second suspension may be cultured for an appropriate length of time to yield a confluency between about 60% and 100%, or between about 70% and 95%, as described above. Provided that a seeding density appropriate for the cells is used (e.g., between about 7500-12500 cells/cm²), a confluency as set forth above may be reached in less than 1 week, less than 6 days, less than 5 days, less than 4 days, or less than 3 days.

Methods of this disclosure may further comprise dissociating a monolayer of cells from a second vessel (e.g., a cell hotel or cell stack), as described above with respect to the monolayer of cells of the first vessel. Accordingly, a third suspension of cells or an expanded suspension (e.g., stem cells) may be obtained after dissociating the monolayer of cells from the second vessel. Indeed, a greater quantity of cells may be present in a third suspension of cells compared to an initial quantity of cells seeded into a first vessel and an expanded quantity of cells in a second vessel. A quantity of cells in the third suspension may exceed a quantity of cells in the first suspension by least 1, 2, 3, 4, or 5 orders of magnitude.

Expansion methods of any aspect this disclosure beginning from a first suspension of cells through a second and/or a third suspension of cells have been optimized for rapidity while still maintaining an overall high stem cell quality. As described above, a quantity of cells in a third suspension may exceed a quantity of cells in a first suspension, such as by 1, 2, 3, 4, or 5 orders of magnitude, and such cells exhibit qualities characteristic of the starting cells, such as in terms of expansion rates, (un)differentiation status, differentiation potential, karyotype, etc.

Methods of this disclosure may further comprise contacting deposited cells (of a first suspension and/or the second suspension) with an attachment supplement. An attachment supplement, whether added prior to, during, or after cells of the applicable suspension become exposed thereto, may facilitate settling and/or substrate attachment. As described above, a cell suspension (that comes into contact with a cell culture medium and attachment settlement) may comprise single cells or clusters/clumps, or a mixture of the two.

As described herein, an attachment supplement is not particularly limited, and may comprise a carrier such as one or more albumin. An attachment supplement may further comprise one or more lipids (or at least one fatty acid) and/or lipid-like substances, as described herein. In one embodiment, an attachment supplement comprises an albumin and one or more fatty acids. In one embodiment, an attachment supplement may be a CloneR™-branded reagent (STEMCELL Technologies).

In any aspect of this disclosure, a concentration of the attachment supplement is dependent on the volume of cells deposited into a first and/or second culture vessel. The attachment supplement may be used at 5%, 10%, 20% or more of the volume of the deposited or seeded cells of the first and/or second cells suspension. In one embodiment, the attachment supplement is at least 10% (v/v) of the volume of a culture medium or a seeding medium. Exemplary attachment supplements may include commercially available albumins, such as AlbuMax™-branded products or the like, whether or not recombinant, and regardless of the species from the albumins are derived or isolated (e.g. human, bovine, etc). Other exemplary attachment supplements may include CloneR™-branded reagents (STEMCELL Technologies).

Where at least one exogenously added extracellular matrix or extracellular matrix protein and/or an attachment supplement comes into contact with cells of a cell suspension, a greater percentage of deposited cells adheres to a wall of the first vessel or the second vessel, respectively, when contacted with the at least one exogenously added extracellular matrix or extracellular matrix protein and/or the attachment supplement than if deposited without coming into contact with the at least one exogenously added extracellular matrix or extracellular matrix protein and/or attachment supplement.

In another aspect, methods of this disclosure may relate to expediting the expansion of cells, such as stem cells (e.g. PSC), optionally in a monolayer. Cells of this disclosure may be expanded in a large-scale cell culture vessel, such as a Cell Factory™ or a CELLSTACK™ (e.g. a “scale-out vessel”).

As described herein, cells of this disclosure are not particularly limited, but preferably are stem cells (e.g. PSC) or progenitor cells.

Methods of this aspect may comprise (a) providing a first suspension of cells (as described herein), (b) depositing cells of the first suspension into a first vessel (as described herein), (c) contacting the deposited cells with at least one exogenously added extracellular matrix or extracellular matrix protein (as described herein), (d) culturing the contacted cells as a monolayer in a cell culture medium (as described herein); (e) dissociating the monolayer and preparing a second suspension of cells (as described herein), (f) repeating at least steps (b) to (d) in a second vessel with the second suspension of cells (as described herein), and (g) yielding greater than 1000-fold expansion in two weeks or less (as described herein).

In such aspect, the nature of the first vessel and the second vessel is as described herein, including with respect to culture surface area differences, and either pre-coating or not pre-coating such vessels with at least one exogenously added extracellular matrix or extracellular matrix protein.

In such aspect, the nature of the at least one exogenously added extracellular matrix or extracellular matrix protein is as described herein, including with respect to type, concentration, and when and how cells of a suspension are contacted therewith. For example, deposited cells may be contacted with at least one exogenously added extracellular matrix or extracellular matrix protein before, after, or at the same time as being deposited into the first or second vessel.

Methods of this aspect, may further comprise dissociating a monolayer from a second vessel to yield an expanded suspension. More particularly, practice of the methods (in any aspect disclosed herein) of this disclosure may yield up to a 100-fold, 200-fold, 500-fold, 1000-fold, or up to a 5000-fold, or higher increase in cells. Thus, a 1000-fold or greater fold expansion may be yielded, such as in two weeks or less, 12 days or less, or about 10 days±1 day.

Methods of this or any aspect may comprise contacting deposited cells with an attachment supplement (as described herein). Cells may come into contact with the attachment supplement prior to, during or after having been deposited into a cell culture vessel. As described herein, cells in contact with an attachment supplement may settle and/or attach to a substrate more rapidly or more firmly relative to settling or attachment rates in the absence of the attachment supplement, and thus such cells may begin to proliferate more rapidly, and thus their expansion may be expedited.

Thus, in another aspect of this disclosure are provided methods of improving cell plating efficiencies of cells to be cultured in monolayer. Methods of this aspect of the disclosure may comprise providing a first suspension of cells, such as in a cell culture medium, depositing cells of the first suspension into a first vessel, culturing the deposited cells as a monolayer in a cell culture medium, dissociating the monolayer and preparing a second suspension of cells, and depositing and culturing cells of the second suspension of cells essentially as described in the foregoing such as to yield greater than 1000-fold expansion in two weeks or less.

As described above, and in any aspect of this disclosure, a greater percentage of deposited cells adheres to a wall of the first vessel or the second vessel, respectively, when contacted with the at least one exogenously added extracellular matrix or extracellular matrix protein than if deposited without coming into contact with the at least one exogenously added extracellular matrix or extracellular matrix protein.

As described above, and in any aspect of this disclosure, a greater percentage of deposited cells adheres to a wall of the first vessel or the second vessel, respectively, when contacted with the at least one exogenously added extracellular matrix or extracellular matrix protein and/or the attachment supplement than if deposited without coming into contact with the at least one exogenously added extracellular matrix or extracellular matrix protein and/or the attachment supplement.

As described above, and in any aspect of this disclosure, greater than 1000-fold expansion is expedited when culturing the deposited cells in contact with the culture medium and the at least one exogenously added extracellular matrix or extracellular matrix protein and/or the attachment supplement in comparison to culturing the deposited cells in contact with the culture medium and not in the presence of the at least one exogenously added extracellular matrix or extracellular matrix protein and/or the attachment supplement.

Methods of any aspect of this disclosure, may comprise combining the at least one exogenously added extracellular matrix or extracellular matrix protein and the culture medium before the deposited cells are contacted with the at least one exogenously added extracellular matrix or extracellular matrix protein.

Methods of any aspect of this disclosure, may comprise spiking the at least one extracellular matrix or extracellular matrix protein into cell culture medium pre-dispensed into the first or second vessel.

In one embodiment, a greater percentage of cells in a first suspension may adhere to a wall of a first vessel when seeded in the presence of a culture medium and an attachment supplement of this disclosure, than if seeded in the culture medium lacking the attachment supplement. In one embodiment, a greater percentage of cells in a second suspension adhere to a wall of a second vessel when seeded in the presence of a culture medium and an attachment supplement of this disclosure than if seeded in the culture medium lacking the attachment supplement.

Methods of any aspect of this disclosure may be amenable to automated culture or performed by automation. In particular, avoiding a requirement to pre-coat cell culture vessels may significantly reduce processing/preparation time, and may also be amenable to processes using liquid-handler robots. Thus, the methods disclosed herein improve scale-up processes of cells, such as by yielding high quantities of high-quality cells in a simplified workflow.

The following non-limiting examples are illustrative of the present disclosure.

### Examples

### Example 1: Maintenance and Harvest of PSCs

Human pluripotent stem cells (PSCs) were maintained in mTeSR™-branded media, such as mTeSR™ Plus or AOF (STEMCELL Technologies) on Corning® Matrigel®. PSCs were passaged as described by the manufacturer, but generally cultures were ready for passage at ~60-80% confluency, and when the majority of colonies were large, compact, and had dense multi-layered centers. Various iPSC lines (SCTi003-A, WLS-1C, H9 and R038) were used in downstream expansion experiments. PSCs could be seeded as single cells or as clumps.

PSC at a confluence of ≥60% (~2×10⁶ viable cells) from one well of a 6-well Matrigel® coated plate were seeded into a T-175 flask. If harvested at a lower confluence, cells from more than 1 well may be required to yield 2×10⁶ viable cells.

To dissociate cells, they were rinsed with PBS and contacted with ~1 ml of a trypsin containing solution, such as TrypLE™ Express (ThermoFisher). The enzyme was inactivated with either 1 ml of mTeSR™ Plus or ACF Enzyme Inhibition Solution (STEMCELL Technologies), and the cells were dislodged by pipetting up and down 1-2 times. The single cell suspension were transferred to a Falcon® tube and pelleted, before being resuspended in 1-2 mL of seeding medium (10% CloneR™ 2 in mTeSR™ Plus) (3.7 mL of CloneR™ 2+33.3 mL of mTeSR™ Plus to prepare 37 mL) and viable cells were counted.

### Example 2: Seeding and Harvesting PSCs as Single Cells in a T-175 Flask

PSCs were maintained in mTeSR™ Plus on hESC-qualified Matrigel™ pre-coated 6 well plate and dissociated essentially as described in Example 1. For plating into a T-175 flask, Matrigel was thawed and added to the seeding medium to achieve a 0.3-0.6% final concentration (e.g., T-175 seeding volume=20 mL, 0.3%×20 mL=0.06 mL Matrigel).

On the day of seeding, 9000 cells/cm² in 35 mL (0.3-0.6% Matrigel) seeding medium was added into a T-175 flask that was not pre-coated with an extracellular matrix. Cells were evenly distributed by shaking the flask back and forth and cultured at 37° C. for 4-5 days. A full medium change with mTeSR™ Plus medium (-Matrigel) was performed on day 1 and on days 3 and 4 after seeding, or could be performed daily.

On day 5 of expansion or when cultures reached 90-95% confluence, the cells were rinsed with PBS and contracted with 12 mL of a trypsin-based solution, such as TryPLE™ Express. An equivalent volume of mTeSR™ Plus or ACF Enzyme Inhibition Solution was added to quench the enzyme, and the cells were dislodged by gently pipetting up and down ~3×. The single cell suspension was transferred to a tube and pelleted, then resuspended in a small volume (~20 mL) of seeding medium, and viable single cells were counted. A number of cells calculated from a 9000 cells/cm² seeding density were used to seed a 10-layer scale-out vessel. PSCs seeded and cultured as described above for 5 days yielded between about 7-10×10⁷ cells.

### Example 3: Seeding and Feeding PSCs in a 10-layer Scale-Out Vessel

To scale-out expansion of STi003-A cells, a cell suspension as obtained in Example 2 was plated in a 10-layer flask, such as a Nunc™ Cell Factory System or Corning™ CELLSTACK™. An appropriate number of STi003-A cells were added into ~1300 ml of seeding medium (10% CloneR™ 2 in mTeSR™ Plus) in a 2 L bottle/container (e.g. 9000 cells/cm^{2×6360} cm² culture surface area =5.724×10⁷ cells).

To monitor cell health and confluence, as a 10-layer scale-out vessel cannot be viewed under a standard bright field microscopy, a “companion flask” such as a T-75 flask was seeded at the same cell density per cm², fed at the same frequency with a proportional volume of mTeSR™ Plus per cm² as the scale-out vessel.

A volume of Matrigel to be spiked into the cell suspension before seeding in the scale-out vessel was calculated as per the formula in Example 2, and the scale-out vessel was seeded by pouring ~1300 mL of cell suspension into an opened port, allowing media to equilibrate to all layers, and placing in an incubator carefully to prevent media from spilling between layers.

Cells in the 10-layer scale-out vessel and the companion T-75 flask were fed on days 1, 3 and 4 post-seeding. Optionally, cells in both containers could be fed daily.

### Example 4: Harvesting PSCs from a 10-Layer Scale-Out Vessel

Cells in the companion T-75 flask of Example 3 were monitored for confluency and when the cells reached 90-95% confluent (day 5-6 post seeding), cells in both containers were harvested.

Cells in the scale-out vessel were rinsed with 200 mL of PBS, incubated with a trypsin-containing solution, such as 200 ml of TrypLE Express or 0.25% Trypsin-EDTA, quenched with 200 mL of ACF Enzyme Inhibition Solution or mTeSR™ Plus, and detached cells were poured into a sterile 1 L bottle/container. Remaining cells in the scale-out vessel were collected in PBS and poured into the same sterile collection bottle. A 50 mL serological pipette was used to triturate the collected cell suspension to ensure dissociation of aggregates into single cells, aliquoted into conical centrifuge tubes, and centrifuged at 300×g for 5 minutes to remove residual dissociation reagent. Cells were resuspended in mTeSR™ Plus, optionally supplemented with 10% CloneR™2, before performing cell counts and differentiation or freezing protocols.

Cells in the companion T-75 flask may be harvested in parallel.

### Example 5: Comparable Morphology and Fold Expansion of PSCs in Pre-Coating Free Methods

Viable fold expansion and morphology of PSCs expanded as described in Examples 3 and 4, or by conventional pre-coating methods were compared.

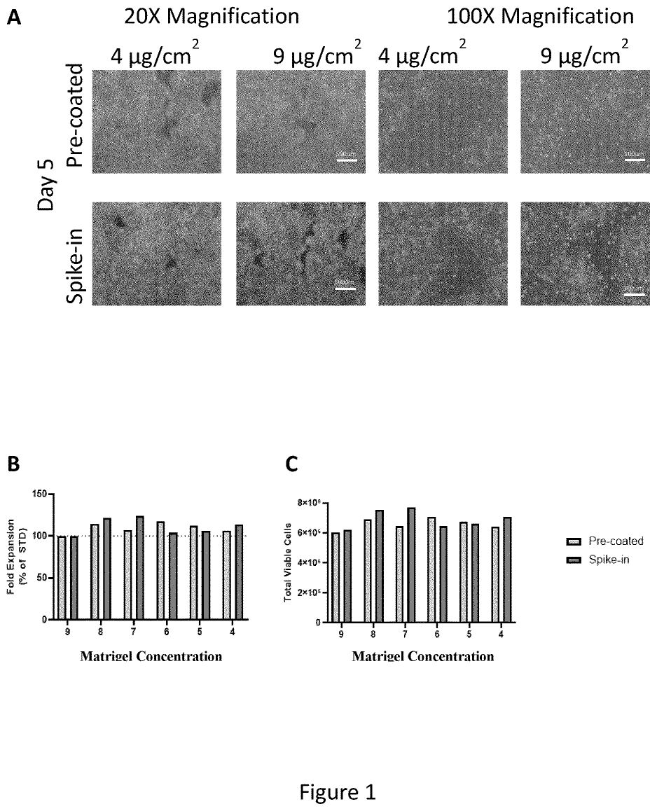

Comparable morphology and confluency were observed for both conditions tested, either at 4 μg/cm² or 9 μg/cm² of Matrigel (FIG. 1A). On day 5, average fold expansion of PSCs in the 9 μg/cm² Matrigel pre-coated control condition and the 9 μg/cm² Matrigel spike-in condition was found to be 8.36 and 8.61 respectively (data not shown).

Moreover, comparable day 5 viable fold expansion of PSCs was observed across multiple Matrigel conditions for both the pre-coated and spike-in protocols (FIG. 1B). Further, the day 5 fold expansion values among day 5 PSCs were comparable at either the standard Matrigel concentration (8.9 μg/cm²) or at 4 μg/cm² (FIG. 1B). The total number of viable cells were also comparable for both pre-coated and spike-in conditions (FIG. 1C).

Overall, the data suggest that the time-consuming process of pre-coating culture vessels can be avoided, and that cells do not appear to be impacted negatively under such conditions.

### Example 6: PSC Expansion in Coating-Free Scale-Out Vessels

Expansion performance of PSCs (iPSC: STi003-A and WLS-1C; ESC: H9) in accordance with Examples 3 and 4 was assessed when starting from an optimized seeding density of 8,000-11,500 cells/cm².

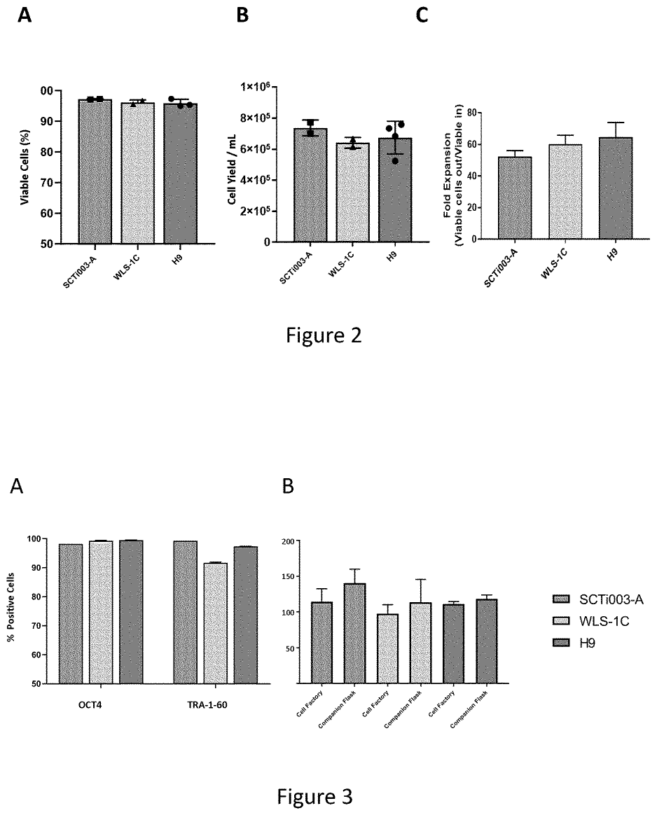

All three PSC lines showed cell viability ≥95% when expanded in a 10-layer scale-out vessel (FIG. 2A), an average of 6.83×10⁵ viable cells/mL (FIG. 2B), and an average of over 3 billion cells (3.5×10⁹ viable PSCs) from the 10-layer scale-out vessel in 5 days, corresponding to fold expansions of about 40-60 times within a period of 5 days (FIG. 2C).

### Example 7: Quality and Attachment of PSCs Expanded in a Scale-Out Vessel

PSC (iPSC: SCTi003-A and WLS-1C; and ESC: H9) quality was quantified by assessing the percentage of cells expressing undifferentiated markers OCT4 and TRA-1-60.

It was observed that ≥90% of cells expanded according to the novel methods outlined in Examples 3 and 4 expressed OCT4 and TRA-1-60 as determined by flow cytometry (Guava EasyCyte) after staining with PE anti-human TRA-1-60-R and Alexa Fluor® 488 anti-Oct4 (Oct3) antibodies (BioLegend) (FIG. 3A), confirming limited or no differentiation during PSC expansion in accordance with methods of this disclosure.

The effect on attachment in subsequent passages after high density cell culture was investigated for three PSC lines (SCTi003-A, WLS-1C and H9). Briefly, single cells harvested from each of a scale-out vessel and a matched companion T-75 flask were seeded with seeding medium (mTeSR™ Plus supplemented with CloneR™ 2) in different wells of a Matrigel-coated 96-well plate (12 wells per condition). After 24 hours of culture each well was tested for percent cell recovery, and approximately 100% recovery of viable cells was observed (FIG. 3B) in all of the three PSC lines tested.

Overall, the results confirm the possibility of rapidly yielding ~3 billion PSC, with ≥90% of the expanded cells in an undifferentiated state, and recovery of essentially all cells.

### Example 8: Viability, Recovery and Fold Expansion of PSC Lines Frozen at High Cell Density

As it may be desirable to bank PSC expanded in scaled-out cultures for subsequent use, the viability, recovery, and fold expansion of post-thaw PSCs was investigated. Suspensions of PSC (SCTi003-A, WLS-1C, and H9) harvested from a scale-out vessel were placed in FreSR-S (STEMCELL Technologies) on ice up to 2 hours (T=0, T=1, and T=2 hours) to simulate the long vialing process required for the large harvest volume.

Harvested PSCs were vialed at 1×10⁷ cells/vial and frozen at −80° C. in Mr. Frosty's after T=0, T=1, and T=2 hours on ice and transferred to LN 2 after 24 hours.

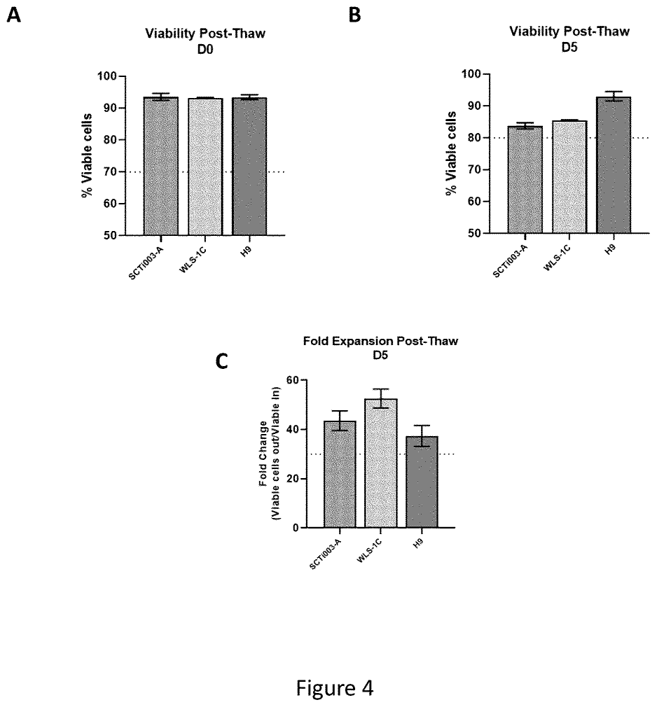

After ≥1 week in LN2, vials from each timepoint were thawed and seeded to assess viability at day 0, and those cells cultured for 5 days were then harvested to determine viability at day 5. It was observed that day 0 post-thaw viability was ≥90% and the day 5 post-thaw viability was ≥80% in all three of the hPSC lines tested (FIG. 4A & FIG. 4B).

After ≥1 week in LN 2, vials from each timepoint were thawed and seeded in replicate to study fold expansion 5 days post-thaw and it was determined that all the three cell lines exhibited greater than 30x fold expansion in 5 days (FIG. 4C).

Overall, cells expanded by the scale-out protocol described herein could be cryopreserved and exhibited marked post-thaw fold expansions.

### Example 9: Fold Expansion of Viable H 9 Cells in Different Spiked-In ECM Proteins

A clump/aggregate suspension of hES line H9 cells was generated from an mTeSR Plus maintained culture and the suspension was counted to determine viable cell number: 3×10⁴ viable H9 cells (corresponding to 1345 clumps) were seeded into 4 replicate wells of 6-well plates.

Matrices tested were Matrigel (Corning), Vitronectin-XF (STEMCELL Technologies), Laminin 511-E8 fragment (Matrixome), and Laminin-521 (BioLamina). For each matrix, there was a pre-coated control and a spike-in condition. Coating concentrations were those recommended by the manufacturer.

For the pre-coated conditions, matrix at the recommended concentration (in μg/cm²) was added to each replicate well and incubated overnight at 4° C. Prior to depositing a cell suspension into the pre-coated wells, the matrix solution was aspirated. For the spike-in conditions, matrix was added directly to the cell suspension prior to deposition into uncoated wells, to a concentration equivalent to the pre-coated control.

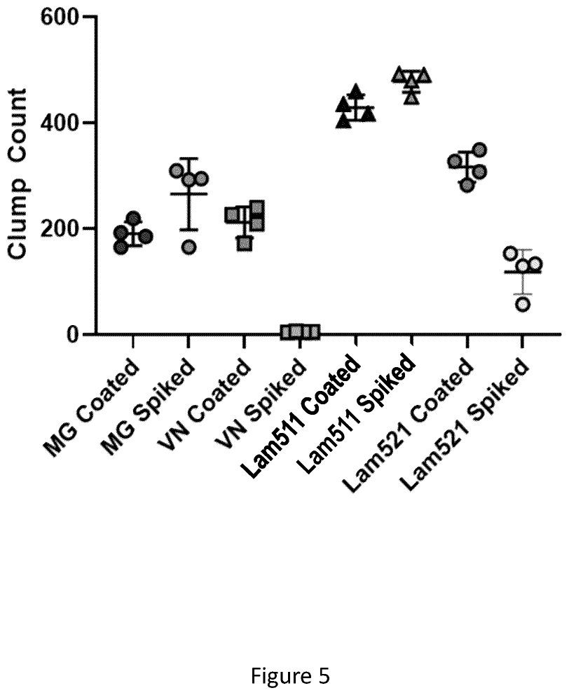

24 hours after seeding, 6-well plates were imaged, and the resulting images were analysed via ImageJ software to quantify the number of hPSC aggregates that had successfully attached to the various matrices. It was observed that the Matrigel spiked-in condition and the Laminin-511 fragment spiked-in condition performed comparably or slightly better than the respective pre-coated conditions (FIG. 5). Moreover, Laminin-521 spike-in condition performed slightly less well compared to the corresponding pre-coating condition.

Overall, the results show the potential for individual extracellular matrix proteins in coating-free cell expansion workflows.

### Example 9.1 hPSC Maintenance Using Coating-Free Laminin

Four human PSC lines (iPSC: SCTi003-A, WLS-1C and STIPS-R038; and ESC: H9) maintained in either mTeSR™ Plus (STEMCELL Technologies) or TeSR™ AOF (STEMCELL Technologies) media were tested for compatibility in coating-free expansion workflows over at least 10 passages. Cells were contacted with a recombinant laminin fragment at a concentration of about 1.25 μg/ml (“CF”), or with the fragment+a surfactant (“CF+S”) at a concentration of about 0.02%. The coating-free conditions were compared to a pre-coated Matrigel control at manufacturer recommended concentrations. Addition of surfactant may improve cell dissociation.

Prior to seeding the pre-coated wells with a cell suspension, the matrix solution was aspirated. For the spike-in conditions, recombinant laminin-511 fragment was added directly to the cell suspension prior to depositing the cells into uncoated wells.

Cell dissociation was performed either essentially as described in Example 1 or using ReLeSR™ PSC selection and passaging reagent (STEMCELL Technologies). Briefly, 1 ml/well of ReLeSR™ was added and aspirated within 1 minute, to expose colonies to a thin film of liquid. The cells were incubated at 37° C. as per manufacturer's instructions. Typically, dissociation for coating-free conditions was 2-3 mins longer than Matrigel™ control conditions but <10 minutes. Scrape dissociation using ReLeSR™ was also tested, wherein a well surface was scraped instead of tapping the plate to dislodge cells. Cells were seeded 8×10³-1×10⁴ live cells per well in a 6- well plate containing 2 ml cell culture medium. Medium was changed throughout the week using restricted feeding schedules as outlined in mTeSR™ Plus and TeSR™-AOF manufacturer's protocols.

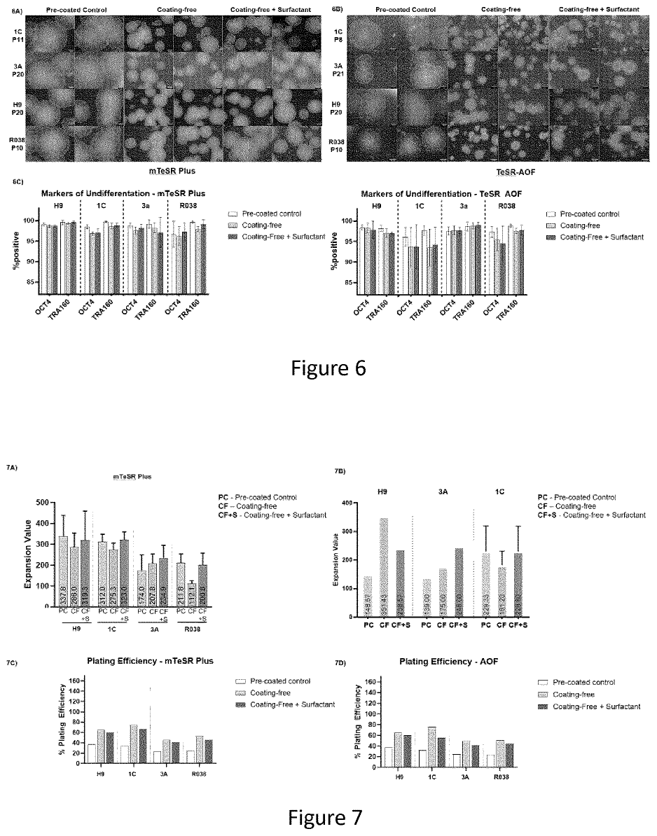

Comparable morphology was observed between pre-coated Matrigel controls and the coating-free conditions (FIGS. 6A and 6B), in either mTeSR™ Plus or TeSR™ AOF media. Round, densely layered colonies, with defined edges were observed in all conditions, and the only difference noted was slightly smaller colonies in coating-free conditions (though confluency was comparable due to more overall colonies overall in coating-free conditions). Thus, the coating-free conditions could support PSC maintenance for at least 10 passages.

The quality of the expanded PSC lines was assessed by quantifying the percentage of cells expressing OCT4 and TRA-1-60, which remained high among all coating-free conditions for all the cell lines tested, in both media formulations (FIG. 6C).

Next, expansion rates were calculated for the 4 cell lines maintained in mTeSR™ Plus and TeSR™ AOF media with either enzymatic dissociation (FIG. 7A) or chemical dissociation of cells (FIG. 7B). With the enzymatic dissociation, comparable expansion rates were observed between the pre-coated and the coating-free conditions (FIG. 7A). When cells were scrape passaged with chemical dissociation, comparable or better expansion rates were observed for the coating-free conditions compared to the pre-coated control (FIG. 7B). The ability to collect all cells using the scrape passaging method demonstrated that all matrices have comparable expansion to the pre-coated Matrigel™ control. Further analysis of plating efficiency of the 4 cell lines cultured in mTeSR™ Plus (FIG. 7C) and TeSR™ AOF (FIG. 7D) media showed a higher plating efficiency for the coating-free conditions compared to the pre-coated Matrigel™ control.

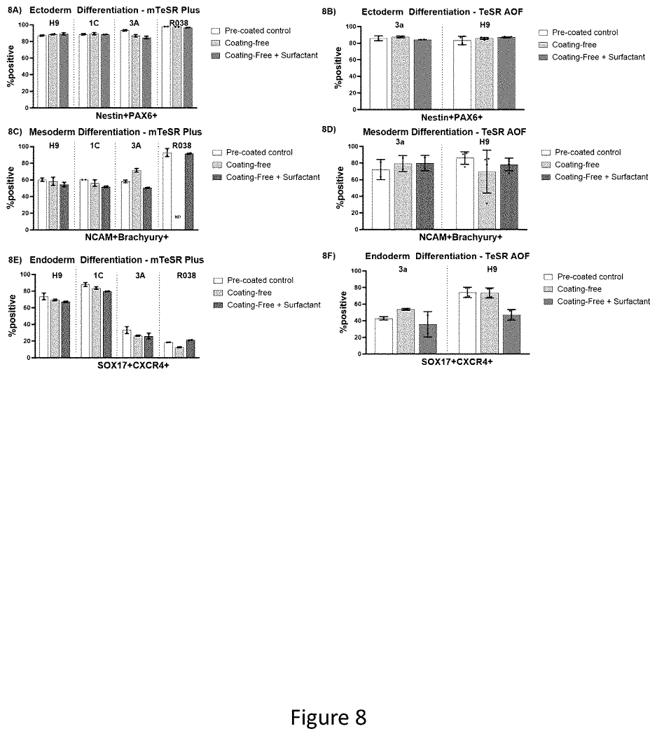

The ability of PSCs expanded in coating-free workflows (CF or CF+S) to differentiate into ectoderm, endoderm and mesoderm lineages (trilineage) was further assessed. Trilineage differentiation of WLS-1C and STIPS-R038 cells (maintained in mTeSR Plus™) and of H9 and SCTi003-A (maintained in mTeSR Plus™ or TeSR™ AOF) were differentiated using the Stemdiff™ Trilineage Differentiation Kit (STEMCELL Technologies) on Matrigel according to the manufacturer's instructions. % positive cells for relevant markers of ectoderm (Nestin, PAX6), endoderm (SOX17, CXCR4) and mesoderm (NCAM, Brachyury) were assessed by flow cytometry. The results in FIG. 8 show that all coating-free expanded PSC exhibited trilineage differentiation, and in many cases equivalent or better than a pre-coated Matrigel™ control.

Further microarray-based karyotype analysis showed for all 4 cell cultures no or limited reportable cytogenetic changes between coating-free and pre-coated conditions (not shown).

### Example 9.2 Porcine PSC (pPSC) Maintenance Using Coating-Free Laminin

pPSCs derived from pig embryos were cultured in a porcine PSC medium comprising Activin A, FGF and XAV939 in 6-well plates. Cells were dissociated using enzymatic methods such as by AccuMAX™, Accutase™ or TrypLE™ as per manufacturer's recommendations, and the cells were then mechanically dissociated by trituration and diluted in the PSC medium. The cells were centrifuged, and resuspended in the PSC medium to create a single cell suspension. The cell suspension was mixed with 10 μM of a RHO/ROCK pathway inhibitor and seeded into a 6-well plate either in contact with pre-coated Laminin 511 (at a concentration of 0.375 mg/ml) or with Laminin 511 (at 50%, 75% or 100%) or 521 (at 1 μg/ml, 2.5 μg/ml, 5 ug/ml, 7.5 μg/ml and 10 μg/ml) added to the PSC medium.

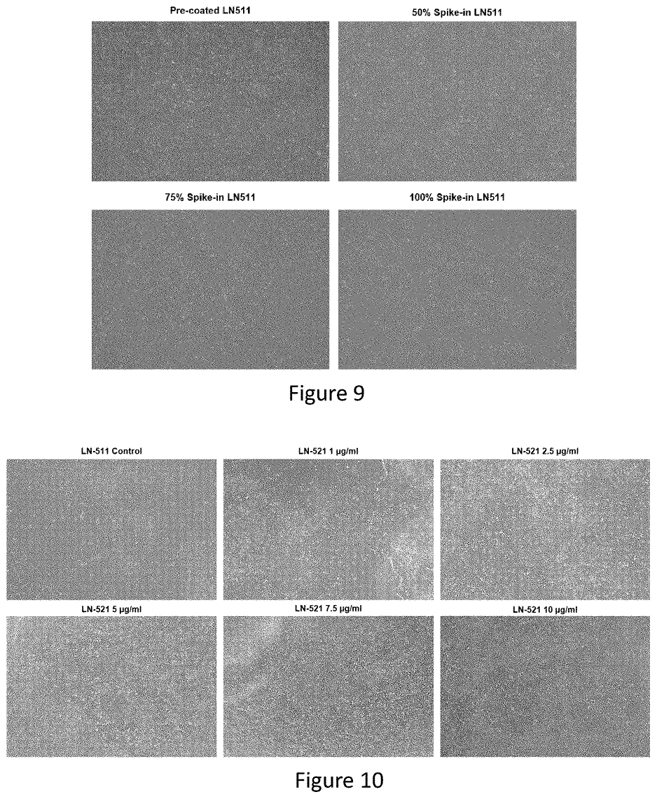

Spiked-in (e.g. coating-free) LN511 displayed morphology and expansion consistent with the LN511 pre-coating control (FIG. 9). LN511 spiked-in at 50% of the pre-coated control concentration (0.375 mg/ml) showed comparable morphology and growth with the LN511 pre-coated control whereas the 75% and 100% spike-in conditions displayed lower number of cells and a slightly different morphology. Spiked-in LN521 displayed morphology and expansion consistent with a LN511 pre-coating control (FIG. 10). LN521 spiked in at lower concentrations (e.g. 1 and 2.5 μg/ml) showed comparable to pre-coated control at the end of passage 0. At higher concentrations (e.g. above 5 μg/ml) of spiked-in LN 521, more loosely packed cells with distinct colony borders were observed (FIG. 10). At subsequent passages, 2.5 μg/ml and 5 μg/ml concentrations of spiked-in LN 521 yielded comparable morphology and growth to a pre-coated LN511 control (data not shown).

The above-described example embodiments of the present disclosure are intended to be illustrative only and in no way limiting. The described embodiments are susceptible to many modifications of composition, details, and order of operation. The invention, rather, is intended to encompass all such modifications within its scope, as defined by the claims, which should be given a broad interpretation consistent with the description as a whole.

1) A method of expanding cells in monolayer, comprising providing a first suspension of cells;
depositing cells of the first suspension into a first vessel;
contacting the deposited cells with at least one exogenously added extracellular matrix or extracellular matrix protein, wherein the first vessel is not pre-coated with the at least one exogenously added extracellular matrix or extracellular matrix protein; and
culturing the contacted cells as a monolayer in a cell culture medium.

2) The method of claim 1, further comprising dissociating the monolayer and preparing a second suspension of cells.

3) The method of claim 2, further comprising
depositing cells of the second suspension into a second vessel;
contacting the deposited cells with the at least one exogenously added extracellular matrix or extracellular matrix protein, wherein the second vessel is not pre-coated with the at least one exogenously added extracellular matrix or extracellular matrix protein; and
culturing the contacted cells as a monolayer in the cell culture medium.

4) (canceled)

5) The method of claim 3, wherein a surface area of the second vessel is greater than a surface area of the first vessel.

6) (canceled)

7) The method of claim 3, further comprising dissociating the monolayer from the second vessel to yield a third suspension.

8) The method of claim 7, wherein a quantity of cells in the second or third suspension exceeds a quantity of cells in the first suspension by at least 3 orders of magnitude.

9) The method of claim 8, wherein the exceeded quantity of cells by at least 3 orders of magnitude elapses two weeks of culture or less.

10) The method of claim 1, wherein the cells are stem cells.

11) (canceled)

12) The method of claim 1, further comprising contacting the deposited cells with an attachment supplement comprising an albumin and one or more fatty acids.

13) The method of claim 1, wherein a greater percentage of deposited cells adheres to a wall of the first vessel or the second vessel, respectively, when contacted with the at least one exogenously added extracellular matrix or extracellular matrix protein than if deposited without coming into contact with the at least one exogenously added extracellular matrix or extracellular matrix protein.

14) The method of claim 1, further comprising combining the at least one exogenously added extracellular matrix or extracellular matrix protein and the culture medium before the deposited cells are contacted with the at least one exogenously added extracellular matrix or extracellular matrix protein.

15) (canceled)

16) An expedited method of expanding cells in a monolayer,
a) providing a first suspension of cells;
b) depositing cells of the first suspension into a first vessel;
c) contacting the deposited cells with at least one exogenously added extracellular matrix or extracellular matrix protein, wherein the first vessel is not pre-coated with the at least one exogenously added extracellular matrix or extracellular matrix protein;
d) culturing the contacted cells as a monolayer in a cell culture medium;
e) dissociating the monolayer and preparing a second suspension of cells;
f) repeating at least steps b) to d) in a second vessel with the second suspension of cells; and
g) yielding greater than 1000-fold expansion in two weeks or less.

17) The method of claim 16, wherein a surface area of the second vessel is greater than a surface area of the first vessel.

18) (canceled)

19) (canceled)

20) The method of claim 16, further comprising dissociating the monolayer from the second vessel to yield an expanded suspension.

21) The method of claim 16, wherein the cells are stem cells.

22) (canceled)

23) The method of claim 16, further comprising contacting the deposited cells with an attachment supplement comprising an albumin and one or more fatty acids.

24) The method of claim 16, wherein a greater percentage of deposited cells adheres to a wall of the first vessel or the second vessel, respectively, when contacted with the at least one exogenously added extracellular matrix or extracellular matrix protein than if deposited without coming into contact with the at least one exogenously added extracellular matrix or extracellular matrix protein.

25) The method of claim 23, wherein a greater percentage of deposited cells adheres to a wall of the first vessel or the second vessel, respectively, when contacted with the at least one exogenously added extracellular matrix or extracellular matrix protein and/or the attachment supplement than if deposited without coming into contact with the at least one exogenously added extracellular matrix or extracellular matrix protein and/or the attachment supplement.

26) The method of claim 25, wherein greater than 1000-fold expansion is expedited when culturing the deposited cells in contact with the culture medium and the at least one exogenously added extracellular matrix or extracellular matrix protein and/or the attachment supplement in comparison to culturing the deposited cells in contact with the culture medium and not in the presence of the at least one exogenously added extracellular matrix or extracellular matrix protein and/or the attachment supplement,

27) The method of claim 1, further comprising combining the at least one exogenously added extracellular matrix or extracellular matrix protein and the culture medium before the deposited cells are contacted with the at least one exogenously added extracellular matrix or extracellular matrix protein.

28) (canceled)

29) (canceled)
